(19) United States
(12) Patent Application Publication
(10) Pub. No.: US 2017/0095534 A1
(43) Pub. Date: Apr. 6, 2017

(54) COMPOSITIONS AND METHODS FOR TREATING OR PREVENTING RADIATION- OR CHEMOTHERAPY-INDUCED PULMONARY DYSFUNCTION

(71) Applicant: Reponex Pharmaceuticals ApS, Hørsholm (DK)

(72) Inventor: Lars Heslet, Gentofte (DK)

(21) Appl. No.: 15/377,867

(22) Filed: Dec. 13, 2016

Related U.S. Application Data
(62) Division of application No. 14/934,713, filed on Nov. 6, 2015, now abandoned.
Application No. 14/934,713 is a continuation of application No. 14/240,573, filed on Feb. 24, 2014, now abandoned.
(60) Provisional application No. 61/528,459, filed on Aug. 29, 2011.

(51) Int. Cl. A61K 38/19; A61K 9/00

(52) U.S. Cl. CPC A61K 38/193; A61K 9/0078

(57) ABSTRACT

Compositions comprising one or more cytokines and methods for their use in inhibiting and/or alleviating effects of radiation therapy and/or chemotherapy and/or acute radiation syndrome in a subject in need thereof are provided.

### FIELD OF THE INVENTION

The present invention relates to compositions of selected cytokines and methods for their use in inhibiting and/or alleviating effects of radiation therapy and/or chemotherapy and/or acute radiation syndrome in a subject in need thereof.

### BACKGROUND OF THE INVENTION

The current radiation threat from the Fukushima power plant accident has prompted rethinking of the contingency plan for prophylaxis and treatment of the Acute Radiation Syndrome (ARS).

The exposure with a high dose radiation induces the so-called Acute Radiation Syndrome (ARS) followed by severe injury to the stem cells, the organs, and the tissues (Fauci et al. Radiation Injury. In: Harrison's Principles of Internal Medicine, 17th Edition. 17th ed. McGraw-Hill Professional; 2008:2559). The subsequent seriously affected patient experiences a reduced immunological defense against exogenous and endogenous factors, such as infection and inflammation, and consequently suffers from invasive infection and organ dysfunction, leading to bone marrow aplasia, which may be relieved by human stem cell transplantation (HSCT) (Hall E J. Acute effects of total-body irradiation. In: Radiobiology for the Radiologist. Fifth. Lippincott Williams & Wilkins; 2000:124-135; Cerveny et al. Acute Radiation Syndrome in Humans. In: Medical Consequences of Nuclear Warfare. Vol 1989. Nuclear Agency/Falls Church: TMM Publications, Office of the Surgeon General; Anno et al. Gy. Health Phys. 1989; 56(6):821-838). In extreme cases the radiation injury may be fatal for the exposed person (Anno et al. Gy. Health Phys. 1989; 56(6):821-838; Mettler et al. Health Phys. 2007; 93(5):462-469; Thongpraparn et al. Australas Phys Eng Sci Med. 2002; 25(4):172-174; Liu et al. J. Radiat. Res. 2008; 49(1):63-69).

All organs may be affected and damaged. However, the airways are reported to have the highest incidence of signs and symptoms and infections. The reason is that the airways are exposed to a double hit injury as the lungs receive both the exposure to gamma irradiation as the rest of the body, and additional potential radiation from inhaled radioactive dust particles. Apart from the reduced host defense other injuries are imposed to the skin like burn injury, and the complications and treatment are equivalent to the care from mild to 3rd degree burns, depending on the radiation dose (Anno et al. Gy. Health Phys. 1989; 56(6):821-838; Mettler et al. Health Phys. 2007; 93(5):462-469; Friesecke et al. Radiat Environ Biophys. 2000; 39(3):213-217; Junk et al. Klin Monbl Augenheilkd. 1999; 215(6):355-360; Belyi et al. Health Phys. 2010; 98(6):876-884).

Management of patients with ARS includes early use of hematopoietic cytokines, antimicrobials, and transfusion support. Recommendations based on radiation dose and physiologic response is made for treatment of the hematopoietic syndrome, and therapy includes systemic treatment with hematopoietic cytokines;

blood transfusion; and, in selected cases, stem-cell transplantation (Waselenko et al. Ann. Intern. Med. 2004; 140(12):1037-1051; Gourmelon et al. Health Phys. 2010; 98(6):825-832; Weisdorf et al. Biol. Blood Marrow Transplant. 2006; 12(6):672-682).

Additional medical management based on the evolution of clinical signs and symptoms includes the use of antimicrobial agents (quinolones, antiviral therapy, and antifungal agents), antiemetic agents, and analgesic agents. Because of the strong psychological impact of a possible radiation exposure, psychosocial support will be required for those exposed, regardless of the dose (Gourmelon et al. Health Phys. 2010; 98(6):825-832; Weisdorf et al. Biol. Blood Marrow Transplant. 2006; 12(6):672-682).

The prevention and management of infection is a mainstay of therapy. There is a quantitative relationship between the degree of neutropenia and the increased risk of infectious complications (Waselenko et al. Ann. Intern. Med. 2004; 140(12):1037-1051; Gourmelon et al. Health Phys. 2010; 98(6):825-832; Weisdorf et al. Biol. Blood Marrow Transplant. 2006; 12(6):672-682). Additional factors including duration of neutropenia, bactericidal functionality of surviving neutrophils, alteration of physical defense barriers, the patient's endogenous microflora, and organisms endemic to the hospital and community also affect treatment choices. As the duration of neutropenia increases, the risk of secondary infections such as invasive mycoses also increases (Fliedner et al. Blood. 1964; 23:471-487).

Growth factors have an important effect in respect to prophylaxis and survival provided that the treatment is administered promptly (Butturini et al. Lancet. 1988; 2(8609):471-475). Systemic or subcutaneous administration of growth factor granulocyte stimulating factor (GM-CSF) in acute radiation injury has become a standard treatment for ARS in the U.S. as GM-CSF increases number and function of granulocytes.

### SUMMARY OF THE INVENTION

As aspect of the present invention relates to a method for inhibiting and/or alleviating effects of radiation therapy and/or chemotherapy and/or acute radiation syndrome in a subject in need thereof, said method comprising administering to the lungs a composition comprising a selected cytokine or a combination thereof.

In one embodiment, the cytokine comprises granulocyte-macrophage colony-stimulating factor (GM-CSF), macrophage colony-stimulating factor (M-CSF), granulocyte colony-stimulating factor (G-CSF), stem cell factor (SCF), and/or an interleukin series (IL-1 to IL-16).

In one embodiment, the composition is administered locally.

In one embodiment, the cytokine is pegylated.

In one embodiment, the composition is administered as a liposomal formulation.

In one embodiment, the composition is administered to a subject suffering from acute radiation syndrome.

In one embodiment, the composition is administered to a subject prior to and/or during radiation therapy.

In one embodiment, the composition is administered to a subject prior to and/or during chemotherapy.

### DESCRIPTION OF THE FIGURES

FIG. 1 provides a diagram of the mechanism of action of GM-CSF following systemic administration either by infusion or subcutaneous dosing. GM-CSF activates the stem cells of neutrocytes and macrophages/monocytes. Consequently these cell lines maturate and proliferate (1). The circulating monocytes (2) become tissue macrophages, which are present both in the bone marrow and in the peripheral organs including the lungs. The monocytes transforms into tissue macrophages in tissues (3). After stimulation the GM-CSF receptors transforms the resting alveolar macrophages into the immunocompetent dendritic cells corresponding to the autocrinic GM-CSF response locally (4), during which process both T-lymphocytes and granulocytes are being recruited from the circulation (5).

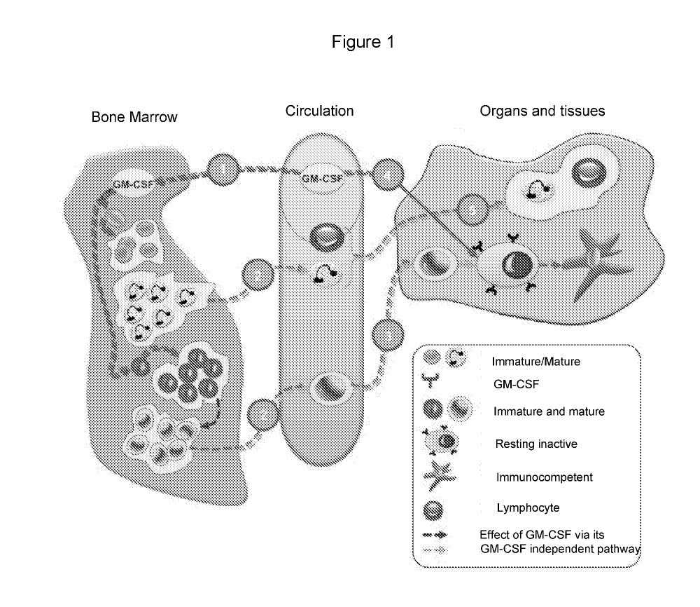

FIGS. 2a and 2b provides a diagram of systemic (FIG. 2a;Ia) versus local administration (FIG. 2B;Ib) of GM-CSF. The lung is a particularly vulnerable vital organ, when exposed to acute radiation irradiation because of the double hit of radiation exposure, i.e. a combined exposure of inhaled particles (P) and from gamma radiation (γ) similar to the rest of the body (A). The lung's host is dependent on its local GM-CSF being expressed by the alveolar cells. After intravenous or subcutaneous administration, GM-CSF does not reach its target in the alveolar space. On the contrary the GM-CSF is sealed off from the airspace due to its water-solubility and molecular size. In order to up-regulate the pulmonary host by activating the resting alveolar macrophages, the GM-CSF has to be inhaled. Due to radiation injury the lung is accordingly exposed to severe dysfunction. As shown, GM-CSF does not penetrate the alveolocapillary membrane either from the blood side to the air side or vice versa.

### DETAILED DESCRIPTION OF THE INVENTION

The lungs have their own host defense system, based on alveolar macrophages. After radiation exposure to the lungs, resting macrophages can no longer be transformed, not even during systemic administration of growth factors/cytokines because G-CSF/GM-CSF does not penetrate the alveoli. Under normal circumstances, locally-produced GM-CSF receptors transform resting macrophages into fully immunocompetent dendritic cells in the sealed-off pulmonary compartment. However, GM-CSF is not expressed in radiation injured tissue due to defervescence of the macrophages.

In order to maintain the macrophage's important role in host defense after radiation exposure, it is necessary to administer the cytokines exogenously in order to uphold the barrier against exogenous and endogenous infections and possibly prevent the potentially lethal systemic infection, which is the main cause of death in ARS.

ARS is a combination of acute injury manifestations that occur after a sufficiently large portion of the body is exposed to a high dose of ionizing radiation. ARS is defined as the signs and symptoms that occur after a whole-body or significant partial-body (60%) exposure of >1 Gy total dose, delivered acutely at a relatively high-dose rate. Such irradiation injury initially affects all organs to some extent, but the timing and extent of the injury manifestations depend upon the type, rate, and dose of radiation received. The percentage of the body that is injured, the dose homogeneity, and the intrinsic radiosensitivity of the exposed individual also influence manifestations. Different ranges of whole-body doses produce different manifestations of injury. The three main ranges that produce the most characteristic manifestations are referred to as the hematological, gastrointestinal, and neurovascular syndromes. These syndromes are, as a rule, produced only with whole-body or near whole-body irradiation by photon or mixed photon/neutron radiation. High-dose injuries to smaller percentages of the body produce local injury effects, but may not cause ARS.

Radiation damage primarily affects proliferating cells because they are the most sensitive to acute effects. The tissues therefore have different sensitivity thresholds for the release of clinical symptoms after radiation. Bone marrow and the intestines have a low threshold caused by fast cellular turnover, whereas muscles and brain cells multiply slowly and are more resistant to radiation. The clinical components of ARS include several subsyndromes, each with a specific trigger sensitivity threshold for the release of clinical symptoms like the hematologic, gastrointestinal, cerebrovascular, and multiorgan/pulmonary dysfunction syndromes.

The most sensitive cells to acute radiation effect are in bone marrow. However, an overlooked fact is that there are other important replicative cells, namely the fixed tissue macrophages in tissue and vital organs. Depending on the absorbed radioactive dose, symptoms appear within hours to weeks, following a predictable clinical course. Four major organ subsystems are known to be of critical significance in the development of ARS: the gastrointestinal system, neurovascular system, hematologic system, and pulmonary system. Evaluation of system-specific signs and symptoms is required for triage of victims, selection of therapy, and determination of prognosis

The present invention provides compositions and methods for inhibiting and/or alleviating effects of radiation therapy and/or chemotherapy and/or acute radiation syndrome in a subject in need thereof.

Compositions of the present invention comprise a selected cytokine such as granulocyte-macrophage colony-stimulating factor (GM-CSF), macrophage colony-stimulating factor (M-CSF), granulocyte colony-stimulating factor (G-CSF), stem cell factor (SCF), and/or an interleukin series (IL-1-IL-16) and combinations thereof. In one embodiment, the composition comprises GM-CSF. In one embodiment, the composition comprises M-CSF. In one embodiment, the composition comprises G-CSF. In one embodiment, the cytokine is pegylated.

For subjects suffering from acute radiation syndrome, the composition is preferably administered by inhalation. Further, the composition is preferably administered in combination with systemic or subcutaneous administration of a selected cytokine such as, but not limited to GM-CSF.

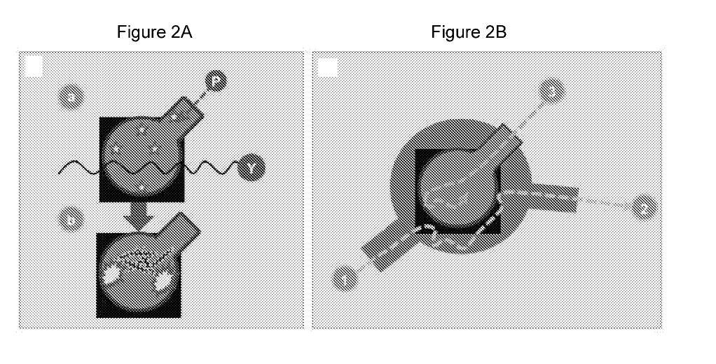

By the administration of GM-CSF systemically, the lung is a specifically exposed to inflammatory and infectious attacks. This in turn leads to acute pulmonary dysfunction, a condition with a very high mortality in itself. Further the lung is specifically vulnerable to radioactive exposure, based on the fact that the lungs host is isolated form the rest of the circulation. As shown in FIG. 2, the alveolo-capillary membrane is “sealed” of from the systemic pool of drugs for protein-like-medicaments. Proteins are, however, soluble and too large to penetrate the membrane. This is the explanation for the acute lung injury after acute radiation exposure, both documented in inhalation of radioactive particles and gamma radiation.

The novel dual treatment plan of the present invention emphasizes the importance of prophylactic treatment with both systemically administered and inhaled adequate doses of GM-CSF in order to ensure a hematologic response in the entire body, including the pulmonary system. Ultimately hematological stem cell transplantation (HSCT) should only be considered provided that the bone marrow aplasia persists after 3 weeks treatment with high doses of GM-CSF without any response in the neutrocyte count, i.e. with no residual hematopoiesis.

The inventors herein believe that the inhaled composition should be an integral part of the anti-radiation intervention in order to maintain the lungs host defense and thus prevent severe pneumonia with endogenous microbiological agents like virus bacteria and fungi. An inhaled composition comprising, for example, GM-CSF should be instituted promptly and concomitantly with the systemic intervention in the anti-radiation therapy regime. In one embodiment, an inhaled high dose of 300 microgram/m2 daily is administered to a subject in need thereof. Alternative doses based upon known efficacy studies and known safety and low toxicity of the drug can be determined routinely by those skilled in the art based upon this disclosure.

GM-CSF for use in the present invention is available through various commercial vendors.

In one embodiment, the GM-CSF is recombinant GM-CSF. In this embodiment, the dose of GM-CSF administered via inhalation can range from about 50 μg/dose/day to 500 μg bid/m² body surface. In one embodiment, the dose of recombinant GM-CSF administered is 300 μg/day.

Doses to be administered for alternative selected cytokines can be determined routinely by those skilled in the art based upon known efficacy studies and known safety and toxicities of the selected cytokines.

For subjects undergoing radiation therapy and/or chemotherapy, compositions of the present invention can be administered subcutaneously or locally. Compositions can be administered prior to, during and/or after radiation therapy and/or chemotherapy to inhibit and/or alleviate effects thereof.

In one embodiment, the composition is administered as a liposomal formulation.

In one embodiment, the subject is a mammal. In one embodiment, the mammal is a human. In one embodiment, the human is a child younger than 15 years of age. In one embodiment, the human is an adult 15 years of age or older.

### Cytokines of the Present Invention

The present invention relates to pulmonary administration of granulocyte-macrophage colony-stimulating factor (GM-CSF), granulocyte colony-stimulating factor (G-CSF), macrophage colony-stimulating factor (M-CSF), stem cell factor (SCF), and/or an interleukin series (IL-1 to IL-16) and combinations thereof, or functional variants or homologues thereof, however prepared (denoted collectively ‘the cytokines’ herein)

The cytokines may be commercially available, e.g. sargramostim (GM-CSF [Leukine®; Immunex, Seattle, Wash.]), filgrastim (G-CSF [Neupogen®; Amgen, Inc, Thousand Oaks, Calif.]) and pegfilgrastim (pegylated G-CSF).

In one embodiment, the composition of the present invention comprise one or more cytokines selected from the group consisting of granulocyte-macrophage colony-stimulating factor (GM-CSF), macrophage colony-stimulating factor (M-CSF), granulocyte colony-stimulating factor (G-CSF), stem cell factor (SCF), and an interleukin (IL-1 to IL-16). One or more in this respect may be 1, 2, 3, 4 or 5 cytokines.

### GM-CSF

Colony-stimulating factors are glycoproteins that stimulate the growth of hematopoietic progenitors and enhance the functional activity of mature effector cells. In brief, at the level of immature cells, CSF's assure the self-renewal of the staminal pool and activate the first stage of hematopoietic differentiation; in the middle stage, when cell proliferation is associated to a progressive acquisition of characteristics of mature cells, they enormously enhance the number of differentiating cells; in the terminal stage they control the circulation and the activation of mature cells.

In a preferred embodiment of the present invention the cytokine to be used is GM-CSF. Mature GM-CSF is a monomeric protein of 127 amino acids with several potential glycosylation sites. The variable degree of glycosylation results in a molecular weight range between 14 kDa and 35 kDa. Non-glycosylated and glycosylated GM-CSF show similar activity in vitro (Cebon et al., 1990). The crystallographic analysis of GM-CSF revealed a barrel-shaped structure composed of four short alpha helices (Diederichs et al., 1991). There are two known sequence variants of GM-CSF. The active form of the GM-CSF protein is found extracellularly as a homodimer in vivo.

GM-CSF exerts its biological activity by binding to its receptor. The most important sites of GM-CSF receptor (GM-CSF-R) expression are on the cell surface of myeloid cells, like alveolar macrophages type I & II, epithelial pulmonary cells and endothelial cells, whereas lymphocytes are GM-CSF-R negative. The native receptor is composed of at least two subunits, alpha and beta. The alpha subunit imparts ligand specificity and binds GM-CSF with nanomolar affinity (Gearing et al., 1989; Gasson et al., 1986). The beta subunit is also part of the interleukin-3 and interleukin-5 receptor complexes and, in association with the GM-CSF receptor alpha subunit and GM-CSF, leads to the formation of a complex with picomolar binding affinity (Hayashida et al., 1990). The binding domains on GM-CSF for the receptor have been mapped: GM-CSF interacts with the beta subunit of its receptor via a very restricted region in the first alpha helix of GM-CSF (Shanafelt et al., 1991b; Shanafelt et al., 1991a; Lopez et al., 1991). Binding to the alpha subunit could be mapped to the third alpha helix, helix C, the initial residues of the loop joining helices C and D, and to the carboxyterminal tail of GM-CSF (Brown et al., 1994).

Formation of the GM-CSF trimeric receptor complex leads to the activation of complex signaling cascades involving molecules of the JAK/STAT families, She, Ras, Raf, the MAP kinases, phosphatidylinositol-3-kinase and NFkB, finally leading to transcription of c-myc, c-fos and c-jun. Activation is mainly induced by the beta subunit of the receptor (Hayashida et al., 1990; Kitamura et al., 1991; Sato et al., 1993). The shared beta subunit is also responsible for the overlapping functions exerted by IL-3, IL-5 and GM-CSF (for review see: de Groot et al., 1998).

Apart from its hemopoietic growth and differentiation stimulating activity, GM-CSF functions especially as a proinflammatory cytokine. Macrophages, e.g. alveolar macrophages type I & II and monocytes as well as neutrophils and eosinophils become activated by GM-CSF, resulting in the release of other cytokines and chemokines, matrix degrading proteases, increased HLA expression and increased expression of cell adhesion molecules or receptors for CC-chemokinesm which in turn, leads to increased chemotaxis of inflammatory cells into inflamed tissue.

Wong et al., Science Vol. 228, pp. 810-815 (1985) and Kaushansky et al., Proc. Natl. Acad. Sci. USA, Vol. 83, pp. 3101-3105 (1986) have described the production of recombinant GM-CSF in mammalian cells. Burgess et al., Blood, Vol. 69, pp. 43-51 (1987) describes the purification of GM-CSF produced in Escherichia coli.

### Functional Homologues of GM-CSF

A functional homologue of GM-CSF is a polypeptide having at least 50% sequence identity with the known and naturally occurring sequence of GM-CSF and has one or more GM-CSF functions, such as the stimulation of the growth and differentiation of hematopoietic precursor cells from various lineages, including granulocytes, macrophages, eosinophils and erythrocytes.

GM-CSF regulates multiple functions of alveolar macrophages (AM). GM-CSF stimulation of AM has been documented to enhance alveolar macrophages selectively respond to noxious ingestants, i.e., stimulation of inflammation during bacterial phagocytosis, nonnoxious ingestants are generally mollified, i.e., antiinflammatory responses during phagocytosis of apoptotic cells. Further AM functions are enhanced by GM-CSF stimulation with subsequent proliferation, differentiation, accumulation and activation. Further these GM-CSF effects also encompasses cell adhesion, improved chemotaxis, Fc-receptor expression, complement- and antibody-mediated phagocytosis, oxidative metabolism, intracellular killing of bacteria, fungi, protozoa, and viruses, cytokine signaling, and antigen presentation. Further GM-CSF enhances defects in AM cell adhesion, pathogen associated molecular pattern receptors, like Toll-like receptors and TLR trans-membranous signaling, surfactant protein and lipid uptake and degradation (Trapnell B C and Whitsett J A. GM-CSF regulates pulmonary surfactant homeostasis and alveolar macrophage-mediated innate host defense. Annu. Rev. Physiol. 2002.64:775-802).

Further GM-CSF interacts with the AM's recognition receptors, the so-called toll like receptors (TLR). GM-CSF is important in the pulmonary host defense in pneumonia due to its interaction with the TLR's participation in the host defense resulting in enhanced clearance of the causative microorganism (Chen G H, Olszewski M A, McDonald R A, Wells J C, Paine R 3rd, Huffnagle G B, Toews G B. Role of granulocyte macrophage colony-stimulating factor in host defense against pulmonary Cryptococcus neoformans infection during murine allergic bronchopulmonary mycosis. Am J Pathol. 2007 March; 170(3):1028-40). Lung has its own innate GM-CSF production, which is reduced in pneumonia and hyperoxia, in relation to high O₂ exposure as seen in, e.g. ventilator associated pneumonia (VAP) contributing impairment of host defense secondary to apoptosis with poor response to infections. The hyperoxic injury seems to be counteracted by activation of alveolar macrophages with GM-CSF (Altemeier W A, Sinclair S E. Hyperoxia in the intensive care unit: why more is not always better. Curr Opin Crit Care. 2007 Feb.; 13(1):73-8. & Baleeiro C E, Christensen P J, Morris S B, Mendez M P, Wilcoxen S E, Paine R. GM-CSF and the impaired pulmonary innate immune response following hyperoxic stress. Am J Physiol Lung Cell Mol Physiol. 2006 December; 291(6):L1246-55. Epub 2006 Aug. 4) with subsequent clearance of P. aeruginosa via expression of the TLR signaling pathway (Baleeiro C E, Christensen P J, Morris S B, Mendez M P, Wilcoxen S E, Paine R. GM-CSF and the impaired pulmonary innate immune response following hyperoxic stress. Am J Physiol Lung Cell Mol Physiol. 2006 December; 291(6):L1246-55. Epub 2006 Aug. 4).

Finally GM-CSF produces in-vitro conversion of AM into immature dendritic cells (DC), which may further be matured with specific agents in respect to activate the homing of matured DC's to a specified receptor or target. (Zobywalski A, Javorovic M, Frankenberger B, Pohla H, Kremmer E, Bigalke I, Schendel D J. Generation of clinical grade dendritic cells with capacity to produce biologically active IL-12p70. J Transl Med. 2007 Apr. 12; 5:18).

Preferably, evolutionary conservation between GM-CSF of different closely related species, e.g. assessed by sequence alignment, can be used to pinpoint the degree of evolutionary pressure on individual residues. Preferably, GM-CSF sequences are compared between species where GM-CSF function is conserved, for example but not limited to mammals including rodents, monkeys and apes. Residues under high selective pressure are more likely to represent essential amino acids that cannot easily be substituted than residues that change between species. It is evident from the above that a reasonable number of modifications or alterations of the human GM-CSF sequence does not interfere with the activity of the GM-CSF molecule according to the invention. Such GM-CSF molecules are herein referred to as functional equivalents of human GM-CSF, and may be such as variants and fragments of native human GM-CSF as described here below.

As used herein the expression “variant” refers to polypeptides or proteins which are homologous to the basic protein, which is suitably human GM-CSF, but which differs from the base sequence from which they are derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide.

A person skilled in the art will know how to make and assess ‘conservative’ amino acid substitutions, by which one amino acid is substituted for another with one or more shared chemical and/or physical characteristics. Conservative amino acid substitutions are less likely to affect the functionality of the protein. Amino acids may be grouped according to shared characteristics. A conservative amino acid substitution is a substitution of one amino acid within a predetermined group of amino acids for another amino acid within the same group, wherein the amino acids within a predetermined groups exhibit similar or substantially similar characteristics. Within the meaning of the term “conservative amino acid substitution” as applied herein, one amino acid may be substituted for another within groups of amino acids characterised by having
i) polar side chains (Asp, Glu, Lys, Arg, His, Asn, Gln, Ser, Thr, Tyr, and Cys,)
ii) non-polar side chains (Gly, Ala, Val, Leu, Ile, Phe, Trp, Pro, and Met)
iii) aliphatic side chains (Gly, Ala Val, Leu, Ile)
iv) cyclic side chains (Phe, Tyr, Trp, His, Pro)
v) aromatic side chains (Phe, Tyr, Trp)
vi) acidic side chains (Asp, Glu)
vii) basic side chains (Lys, Arg, His)
viii) amide side chains (Asn, Gln)
ix) hydroxy side chains (Ser, Thr)
x) sulphor-containing side chains (Cys, Met), and/or
xi) amino acids being monoamino-dicarboxylic acids or monoamino-monocarboxylic-monoamidocarboxylic acids (Asp, Glu, Asn, Gln).

A functional homologue within the scope of the present invention is a polypeptide that exhibits at least 50% sequence identity with human GM-CSF, preferably at least 60%, 70% sequence identity preferably functional homologues have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human GM-CSF.

Sequence identity can be calculated using a number of well-known algorithms and applying a number of different gap penalties. Any sequence alignment algorithm, such as but not limited to FASTA, BLAST, or GETSEQ may be used for searching homologues and calculating sequence identity. Moreover, when appropriate any commonly known substitution matrix, such as but not limited to PAM, BLOSSUM or PSSM matrices, may be applied with the search algorithm. For example, a PSSM (position specific scoring matrix) may be applied via the PSI-BLAST program. Moreover, sequence alignments may be performed using a range of penalties for gap opening and extension. For example, the BLAST algorithm may be used with a gap opening penalty in the range 5-12, and a gap extension penalty in the range 1-2.

Accordingly, a variant or a fragment thereof according to the invention may comprise, within the same variant of the sequence or fragments thereof, or among different variants of the sequence or fragments thereof, at least one substitution, such as a plurality of substitutions introduced independently of one another.

It is clear from the above outline that the same variant or fragment thereof may comprise more than one conservative amino acid substitution from more than one group of conservative amino acids as defined herein above.

Aside from the twenty standard amino acids and two special amino acids, selenocysteine and pyrrolysine, there are a vast number of “nonstandard amino acids” which are not incorporated into protein in vivo. Examples of nonstandard amino acids include the sulfur-containing taurine and the neurotransmitters GABA and dopamine. Other examples are lanthionine, 2-Aminoisobutyric acid, and dehydroalanine. Further non standard amino are ornithine and citrulline.

Non-standard amino acids are usually formed through modifications to standard amino acids. For example, taurine can be formed by the decarboxylation of cysteine, while dopamine is synthesized from tyrosine and hydroxyproline is made by a posttranslational modification of proline (common in collagen). Examples of non-natural amino acids are those listed e.g. in 37 C.F.R. section 1.822(b)(4), all of which are incorporated herein by reference.

Both standard and non standard amino acid residues described herein can be in the “D” or “L” isomeric form.

It is contemplated that a functional equivalent according to the invention may comprise any amino acid including non-standard amino acids. In preferred embodiments a functional equivalent comprises only standard amino acids.

The standard and/or non-standard amino acids may be linked by peptide bonds or by non-peptide bonds. The term peptide also embraces post-translational modifications introduced by chemical or enzyme-catalyzed reactions, as are known in the art. Such post-translational modifications can be introduced prior to partitioning, if desired. Amino acids as specified herein will preferentially be in the L-stereoisomeric form. Amino acid analogs can be employed instead of the 20 naturally-occurring amino acids. Several such analogs are known, including fluorophenylalanine, norleucine, azetidine-2-carboxylic acid, S-aminoethyl cysteine, 4-methyl tryptophan and the like.

Suitably variants will be at least 60% identical, preferably at least 70% and accordingly, variants preferably have at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the predetermined sequence of human GM-CSF.

Functional equivalents may further comprise chemical modifications such as ubiquitination, labeling (e.g., with radionuclides, various enzymes, etc.), pegylation (derivatization with polyethylene glycol), or by insertion (or substitution by chemical synthesis) of amino acids (amino acids) such as ornithine, which do not normally occur in human proteins.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encom-passed within the present invention. Functional equivalents also comprise glycosylated and covalent or aggregative conjugates formed with the same molecules, including dimers or unrelated chemical moieties. Such functional equivalents are prepared by linkage of functionalities to groups which are found in fragment including at any one or both of the N- and C-termini, by means known in the art.

The term “fragment thereof” may refer to any portion of the given amino acid sequence. Fragments may comprise more than one portion from within the full-length protein, joined together. Suitable fragments may be deletion or addition mutants. The addition of at least one amino acid may be an addition of from preferably 2 to 250 amino acids, such as from 10 to 20 amino acids, for example from 20 to 30 amino acids, such as from 40 to 50 amino acids. Fragments may include small regions from the protein or combinations of these.

Suitable fragments may be deletion or addition mutants. The addition or deletion of at least one amino acid may be an addition or deletion of from preferably 2 to 250 amino acids, such as from 10 to 20 amino acids, for example from 20 to 30 amino acids, such as from 40 to 50 amino acids. The deletion and/or the addition may—independently of one another—be a deletion and/or an addition within a sequence and/or at the end of a sequence.

Deletion mutants suitably comprise at least 20 or 40 consecutive amino acid and more preferably at least 80 or 100 consecutive amino acids in length. Accordingly such a fragment may be a shorter sequence of the sequence of human GM-CSF comprising at least 20 consecutive amino acids, for example at least 30 consecutive amino acids, such as at least 40 consecutive amino acids, for example at least 50 consecutive amino acids, such as at least 60 consecutive amino acids, for example at least 70 consecutive amino acids, such as at least 80 consecutive amino acids, for example at least 90 consecutive amino acids, such as at least 95 consecutive amino acids, such as at least 100 consecutive amino acids, such as at least 105 amino acids, for example at least 110 consecutive amino acids, such as at least 115 consecutive amino acids, for example at least 120 consecutive amino acids, wherein said deletion mutants preferably has at least 75% sequence identity, for example at least 80% sequence identity, such as at least 85% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with human GM-CSF.

It is preferred that functional homologues of GM-CSF comprises at the most 500, more preferably at the most 400, even more preferably at the most 300, yet more preferably at the most 200, such as at the most 175, for example at the most 160, such as at the most 150 amino acids, for example at the most 144 amino acids.

The term “fragment thereof” may refer to any portion of the given amino acid sequence. Fragments may comprise more than one portion from within the full-length protein, joined together. Portions will suitably comprise at least 5 and preferably at least 10 consecutive amino acids from the basic sequence. They may include small regions from the protein or combinations of these.

There are two known variants of human GM-CSF; a T1151 substitution in variant 1 and a 1117T substitution in variant 2. Accordingly, in one embodiment of the invention functional homologues of GM-CSF comprises a sequence with high sequence identity to human GM-CSF NO: 1 or any of the splice variants.

Analogs of GM-CSF are for example described in U.S. Pat. Nos. 5,229,496, 5,393,870, and 5,391,485 to Deeley, et al. Such analogues are also functional equivalents comprised within the present invention.

In one embodiment GM-CSF is used according to the present invention in homo- or heteromeric form. Homo- and heteromeric forms of GM-CSF may comprise one or more GM-CSF monomers or functional homologous of GM-CSF as defined herein above. Homo- and heteromers include dimers, trimers, tetramers, pentamers, septamers, heptamers, octamers, nonamers and decamers.

In one embodiment, a homodimer, trimer or tetramer of GM-CSF is used.

The protein sequence of GM-CSF of Homo Sapiens
(SEQ ID NO: 1):
MWLQSLLLLG TVACSISAPA RSPSPSTQPW EHVNAIQEAR
RLLNLSRDTA AEMNETVEVI SEMFDLQEPT CLQTRLELYK
QGLRGSLTKL KGPLTMMASH YKQHCPPTPE TSCATQIITF
ESFKENLKDF LLVIPFDCWE PVQE

### C-CSF

Granulocyte colony-stimulating factor (G-CSF or GCSF) is a colony-stimulating factor hormone. G-CSF is also known as colony-stimulating factor 3 (CSF 3). It is a glycoprotein, growth factor and cytokine produced by a number of different tissues to stimulate the bone marrow to produce granulocytes and stem cells. G-CSF then stimulates the bone marrow to release them into the blood.

The G-CSF-receptor is present on precursor cells in the bone marrow, and, in response to stimulation by G-CSF, initiates proliferation and differentiation into mature granulocytes. G-CSF stimulates the survival, proliferation, differentiation, and function of neutrophil precursors and mature neutrophils. G-CSF is produced by endothelium, macrophages, and a number of other immune cells. The natural human glycoprotein exists in two forms, a 174- and 180-amino-acid-long protein of molecular weight 19,600 grams per mole. The more-abundant and more-active 174-amino acid form has been used in the development of pharmaceutical products by recombinant DNA (rDNA) technology.

The recombinant human G-CSF synthesised in an E. coli expression system is called filgrastim. The structure of filgrastim differs slightly from the structure of the natural glycoprotein. Filgrastim (Neupogen) and PEG-filgrastim (Neulasta) are two commercially-available forms of rhG-CSF (recombinant human G-CSF). The PEG (polyethylene glycol) form has a much longer half-life, reducing the necessity of daily injections.

Another form of recombinant human G-CSF called lenograstim is synthesised in Chinese Hamster Ovary cells (CHO cells). As this is a mammalian cell expression system, lenograstim is indistinguishable from the 174-amino acid natural human G-CSF.

### Recombinant Production

One or more of the cytokines of the present invention; including granulocyte-macrophage colony-stimulating factor (GM-CSF), granulocyte colony-stimulating factor (G-CSF), macrophage colony-stimulating factor (M-CSF), stem cell factor (SCF), and/or an interleukin series (IL-1 to IL-16) and combinations thereof, or functional variants or homologues thereof, can be produced in various ways, such as isolation from for example human or animal serum or from expression in cells, such as prokaryotic cells, yeast cells, insect cells, mammalian cells or in cell-free systems. GM-CSF is preferred.

In one embodiment of the invention, the cytokine is produced recombinantly by host cells. Thus, in one aspect of the present invention, the cytokine is produced by host cells comprising a first nucleic acid sequence encoding the cytokine operably associated with a second nucleic acid capable of directing expression in said host cells.

The second nucleic acid sequence may thus comprise or even consist of a promoter that will direct the expression of protein of interest in said cells. A skilled person will be readily capable of identifying useful second nucleic acid sequence for use in a given host cell.

The process of producing a recombinant cytokine in general comprises the steps of:
providing a host cell
preparing a gene expression construct comprising a first nucleic acid encoding the cytokine operably linked to a second nucleic acid capable of directing expression of said protein of interest in the host cell
transforming the host cell with the construct,
cultivating the host cell, thereby obtaining expression of the cytokine.

The recombinant cytokine thus produced may be isolated by any conventional method, such as any of the methods for protein isolation described herein below. The skilled person will be able to identify a suitable protein isolation steps for purifying the cytokine.

In one embodiment of the invention, the recombinantly produced cytokine is excreted by the host cells. When the cytokine is excreted the process of producing a recombinant protein of interest may comprise the steps of
providing a host cell
preparing a gene expression construct comprising a first nucleic acid encoding the cytokine operably linked to a second nucleic acid capable of directing expression of said protein of interest in said host cell
transforming said host cell with the construct,
cultivating the host cell, thereby obtaining expression of the cytokine and secretion of the cytokine into the culture medium,
thereby obtaining culture medium comprising the cytokine.

The composition comprising the cytokine and nucleic acids may thus in this embodiment of the invention be the culture medium or a composition prepared from the culture medium.

In another embodiment of the invention said composition is an extract prepared from animals, parts thereof or cells or an isolated fraction of such an extract.

In an embodiment of the invention, the cytokine is recombinantly produced in vitro in host cells and is isolated from cell lysate, cell extract or from tissue culture supernatant. In a more preferred embodiment the cytokine is produced by host cells that are modified in such a way that they express the relevant cytokine. In an even more preferred embodiment of the invention said host cells are transformed to produce and excrete the relevant cytokine.

### Administration

An effective amount of a cytokine according to the present invention, including granulocyte-macrophage colony-stimulating factor (GM-CSF), granulocyte colony-stimulating factor (G-CSF), macrophage colony-stimulating factor (M-CSF), stem cell factor (SCF), and/or an interleukin series (IL-1 to IL-16) and combinations thereof, or functional variants or homologues thereof, are preferably administered by pulmonary or airway administration including intratracheal, intrabronchial or bronchio-alveolar administration.

Methods of intratracheal, intrabronchial or bronchio-alveolar administration include, but are not limited to, spraying, lavage, inhalation, flushing or installation, using as fluid a physiologically acceptable composition in which the cytokine have been dissolved. When used herein the terms “intratracheal, intrabronchial or intraalveolar administration” include all forms of such administration whereby the cytokine is applied into the trachea, the bronchi or the alveoli, respectively, whether by the instillation of a solution of the cytokine, by the cytokine in a powder form, or by allowing GM-CSF to reach the relevant part of the airway by inhalation of the cytokine as an aerosolized or nebulized solution or suspension or inhaled powder or gel, with or without added stabilizers or other excipients.

Methods of intrabronchial/alveolar administration include, but are not limited to, bronchoalveolar lavage (BAL) according to methods well known to those skilled in the art, using as a lavage fluid a physiologically acceptable composition in which the cytokine has been dissolved or indeed by any other effective form of intrabronchial administration including the use of inhaled powders containing the cytokine in dry form, with or without excipients, or the direct application of the cytokine, in solution or suspension or powder form during bronchoscopy. Methods for intratracheal administration include, but are not limited to, blind tracheal washing with a similar solution of dissolved cytokine or a cytokine suspension, or the inhalation of nebulized fluid droplets containing dissolved cytokine or a cytokine suspension obtained by use of any nebulizing apparatus adequate for this purpose.

Preferably, said cytokine is to be administered to the air-filled spaces of the lungs.

In another embodiment, intratracheal, intrabronchial or intraalveolar administration does not include inhalation of the product but the instillation or application of a solution of the cytokine or a powder or a gel containing the cytokine into the trachea or lower airways.

Other preferred methods of administration may include using the following devices:
1. Pressurized nebulizers using compressed air/oxygen mixture
2. Ultrasonic nebulizers
3. Electronic micropump nebulizers (e.g. Aeroneb Professional Nebulizer)
4. Metered dose inhaler (MDI)
5. Dry powder inhaler systems (DPI),

The aerosol may be delivered by via a) facemasks or b) via endrotracheal tubes in intubated patients during mechanical ventilation (device 1, 2 and 3). The devices 4 and 5 can also be used by the patient without assistance provided that the patient is able to self-activate the aerosol device.

Preferred concentrations for a solution comprising a cytokine according to the present invention and/or functional homologues or variants thereof are in the range of 0.1 μg to 10000 μg active ingredient per ml solution. The suitable concentrations are often in the range of from 0.1 μg to 5000 μg per ml solution, such as in the range of from about 0.1 μg to 3000 μg per ml solution, and especially in the range of from about 0.1 μg to 1000 μg per ml solution, such as in the range of from about 0.1 μg to 250 μg per ml solution. A preferred concentration would be from about 0.1 to about 5.0 mg, preferably from about 0.3 mg to about 3.0 mg, such as from about 0.5 to about 1.5 mg and especially in the range from 0.8 to 1.0 mg per ml solution.

### Pharmaceutical Composition

Pharmaceutical compositions or formulations for use in the present invention include a cytokine according to the present invention selected from granulocyte-macrophage colony-stimulating factor (GM-CSF), granulocyte colony-stimulating factor (G-CSF), macrophage colony-stimulating factor (M-CSF), stem cell factor (SCF), and/or an interleukin series (IL-1 to IL-16) and combinations thereof, or functional variants or homologues thereof, preferably dissolved in a pharmaceutically acceptable carrier, preferably an aqueous carrier or diluent, or carried to the lower airways as a pegylated preparation or as a liposomal or nanoparticle preparation administered as an aerosol via inhalation, or as a lavage fluid administered via a bronchoscope as a bronchoalveloar lavage or as a blind intratracheal wash or lavage. A variety of aqueous carriers may be used, including, but not limited to 0.9% saline, buffered saline, physiologically compatible buffers and the like. The compositions may be sterilized by conventional techniques well known to those skilled in the art. The resulting aqueous solutions may be packaged for use or filtered under aseptic conditions and freeze-dried, the freeze-dried preparation being dissolved in a sterile aqueous solution prior to administration

In one embodiment a freeze-dried cytokine preparation may be pre-packaged for example in single dose units. In an even more preferred embodiment the single dose unit is adjusted to the patient.

The compositions may contain pharmaceutically acceptable auxiliary substances or adjuvants, including, without limitation, pH adjusting and buffering agents and/or tonicity adjusting agents, such as, for example, sodium acetate, sodium lactate, sodium chloride, potassium chloride, calcium chloride, etc.

The formulations may contain pharmaceutically acceptable carriers and excipients including microspheres, liposomes, microcapsules, nanoparticles or the like.

Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine] (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipids or modifiers of liposomes are preferred e.g. in combination with cholesterol and/or phosphatidylcholine.

Long-circulating liposomes are characterized by their ability to extravasate at body sites where the permeability of the vascular wall is increased. The most popular way of producing long-circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar Lipids, Inc, Alabaster, Ala. Additionally, the liposome suspension may include lipid-protective agents which protect lipids against free-radical and lipid-peroxidative damage on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In some cases, it will be advantageous to include a compound, which promotes delivery of the active substance to its target.

### Dose

By “effective amount” of a cytokine according to the present invention, selected from granulocyte-macrophage colony-stimulating factor (GM-CSF), granulocyte colony-stimulating factor (G-CSF), macrophage colony-stimulating factor (M-CSF), stem cell factor (SCF), and/or an interleukin series (IL-1 to IL-16) and combinations thereof, or functional variants or homologues thereof, it is meant a dose, which, when administered to a patient in need thereof, via pulmonary administration, achieves a concentration in the subject's airways which has a beneficial effect on radiation- or chemotherapeutic effects, i.e. by alleviating and/or preventing symptoms of radiation, especially on the lungs.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are per kilo body weight normally of the order of several hundred μg active ingredient per administration with a preferred range of from about 0.1 μg to 10000 μg per kilo body weight. Doses expected to provide an effective amount of the relevant cytokines are often in the range of from 0.1 μg to 5000 μg per kilo body weight, such as in the range of from about 0.1 μg to 3000 μg per kilo body weight, and especially in the range of from about 0.1 μg to 1000 μg per kilo body weight, preferably in the range of 5 μg to 1000 μg, even more preferred about 100 μg to about 800 μg administered via inhalation once, twice or three times daily.

Suitable daily dosage ranges are per kilo body weight per day normally of the order of several hundred μg active ingredient per day with a preferred range of from about 0.1 μg to 10000 μg per kilo body weight per day. The suitable dosages are often in the range of from 0.1 μg to 5000 μg per kilo body weight per day, such as in the range of from about 0.1 μg to 3000 μg per kilo body weight per day, and especially in the range of from about 0.1 μg to 1000 μg per kilo body weight per day.

GM-CSF may e.g. be administered by inhalation to a patient suffering from moderate to severe asthma in a dose ranging from about 10 to 1000 μg per dose, such as 50-100, 100-200, 200-300, 300-400, 400-500, 500-600, 600-700, 700-800, 800-900, 900-1000 μg per dose, each dose being administered once a day, twice a day, three times a day, four times a day, five times a day or six times a day.

Duration of dosing will typically range from 1 day to about 4 months, such as in the range of 1 day to 2 days, for example 2 days to 3 days, such as in the range of 3 days to 4 days, for example 4-5 days, such as 5-6 days, for example 6-7 days, such as one week to two weeks, for example two to four weeks, such as one month to two months, for example 2 to 4 months.

### Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging comprising from 1 to 7 dosage units, thereby having dosage units for one or more days, or from 7 to 21 dosage units, or multiples thereof, thereby having dosage units for one week of administration or several weeks of administration.

The dosage units can be as defined above. The medical packaging may be in any suitable form for intratracheal, intrabronchial or intraalveolar administration. In a preferred embodiment the packaging is in the form of a vial, ampule, tube, blister pack, cartridge or capsule.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition.

Even more preferably a freeze-dried preparation may be pre-packaged for example in single dose units. In an even more preferred embodiment the single dose unit is adjusted to the patient.

### Indications

It is an aspect of the present invention to provide a composition comprising granulocyte-macrophage colony-stimulating factor (GM-CSF), or a functional variant or homologue thereof, for use in the treatment, prevention or alleviation of radiation-induced or chemotherapeutic-induced pulmonary dysfunction, wherein said cytokine is to be administered locally by pulmonary administration.

It is also an aspect of the present invention to provide use of a composition comprising granulocyte-macrophage colony-stimulating factor (GM-CSF), or a functional variant or homologue thereof, for manufacture of a medicament for the treatment, prevention or alleviation of radiation-induced or chemotherapeutic-induced pulmonary dysfunction, wherein said cytokine is to be administered locally by pulmonary administration.

Prevention may be equivalent to reducing risk of acquiring.

In one embodiment, the radiation-induced or chemotherapeutic-induced pulmonary dysfunction is equivalent to and/or causes a reduced pulmonary immunological host defense against pulmonary infections.

In one embodiment the pulmonary administered GM-CSF, or a functional variant or homologue thereof, is to be administered in combination with systemic and/or subcutaneous administration of GM-CSF.

### Causes of Pulmonary Dysfunction—Irradiation

In one embodiment, there is provided a composition comprising granulocyte-macrophage colony-stimulating factor (GM-CSF), or a functional variant or homologue thereof, for use in the treatment, prevention or alleviation of radiation-induced pulmonary dysfunction, wherein said cytokine is to be administered locally by pulmonary administration, and wherein said radiation-induced pulmonary dysfunction is due to acute radiation syndrome (ARS).

In another embodiment, the present invention provides a composition comprising granulocyte-macrophage colony-stimulating factor (GM-CSF), or a functional variant or homologue thereof, for use in the treatment, prevention or alleviation of radiation-induced pulmonary dysfunction, wherein said cytokine is to be administered locally by pulmonary administration, and wherein said radiation-induced pulmonary dysfunction is due to radiation therapy.

Radiation therapy, radiation oncology, therapeutic radiation or radiotherapy is the medical use of ionizing radiation, generally as part of cancer treatment to control or kill malignant cells. Radiation therapy may be curative in a number of types of cancer if they are localized to one area of the body. It may also be used as part of curative therapy, to prevent tumor recurrence after surgery to remove a primary malignant tumor (for example, early stages of breast cancer). Radiation therapy is synergistic with chemotherapy, and has been used before, during, and after chemotherapy in susceptible cancers.

Radiation therapy is commonly applied to the cancerous tumor because of its ability to control cell growth. Ionizing radiation works by damaging the DNA of exposed tissue leading to cellular death. To spare normal tissues (such as skin or organs which radiation must pass through in order to treat the tumor), shaped radiation beams are aimed from several angles of exposure to intersect at the tumor, providing a much larger absorbed dose there than in the surrounding, healthy tissue. Besides the tumour itself, the radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion. These uncertainties can be caused by internal movement (for example, respiration and bladder filling) and movement of external skin marks relative to the tumor position.

Radiation oncology is the medical specialty concerned with prescribing radiation, and is distinct from radiology, the use of radiation in medical imaging and diagnosis. Radiation may be prescribed by a radiation oncologist with intent to cure (“curative”) or for adjuvant therapy. It may also be used as palliative treatment (where cure is not possible and the aim is for local disease control or symptomatic relief) or as therapeutic treatment (where the therapy has survival benefit and it can be curative). It is also common to combine radiation therapy with surgery, chemotherapy, hormone therapy, immunotherapy or some mixture of the four. Most common cancer types can be treated with radiation therapy in some way. The precise treatment intent (curative, adjuvant, neoadjuvant, therapeutic, or palliative) will depend on the tumor type, location, and stage, as well as the general health of the patient.

The amount of radiation used in photon radiation therapy is measured in gray (Gy), and varies depending on the type and stage of cancer being treated. For curative cases, the typical dose for a solid epithelial tumor ranges from 60 to 80 Gy, while lymphomas are treated with 20 to 40 Gy. Preventative (adjuvant) doses are typically around 45-60 Gy in 1.8-2 Gy fractions (for breast, head, and neck cancers.). Many other factors are considered by radiation oncologists when selecting a dose, including whether the patient is receiving chemotherapy, patient comorbidities, whether radiation therapy is being administered before or after surgery, and the degree of success of surgery.

Total body irradiation (TBI) is a radiation therapy technique used to prepare the body to receive a bone marrow transplant. Brachytherapy, in which a radiation source is placed inside or next to the area requiring treatment, is another form of radiation therapy that minimizes exposure to healthy tissue during procedures to treat cancers of the breast, prostate and other organs.

Radiation therapy has several applications in non-malignant conditions, such as the treatment of trigeminal neuralgia, acoustic neuromas, severe thyroid eye disease, pterygium, pigmented villonodular synovitis, and prevention of keloid scar growth, vascular restenosis, and heterotopic ossification. The use of radiation therapy in non-malignant conditions is limited partly by worries about the risk of radiation-induced cancers.

Historically, the three main divisions of radiation therapy are external beam radiation therapy (EBRT or XRT) or teletherapy, brachytherapy or sealed source radiation therapy, and systemic radioisotope therapy or unsealed source radiotherapy.

In one embodiment, said radiation therapy is targeted at the thorax and/or the lungs. In one embodiment, said radiation therapy targets cancerous tissues of the body, such as cancers of the thorax and/or the lungs.

In one embodiment, said radiation therapy targets a lung cancer of any type, including small-cell lung cancer, non-small-cell lung cancer, pulmonary metastasis of other cancers (e.g. breast cancer, prostate cancer), lymphomas including Hodgkins and non-Hodgkins lymphoma (follicular lymphoma), cancers of the lung pleura (mesothelioma) and/or other cancers in the thoracic cage.

### Causes of Pulmonary Dysfunction—Chemotherapy

In another embodiment, there is provided a composition comprising granulocyte-macrophage colony-stimulating factor (GM-CSF), or a functional variant or homologue thereof, for use in the treatment, prevention or alleviation of chemotherapy-induced pulmonary dysfunction, wherein said cytokine is to be administered locally by pulmonary administration.

Chemotherapy is the treatment of cancer with an antineoplastic drug or with a combination of such drugs into a standardized treatment regimen. Certain chemotherapy agents also have a role in the treatment of other conditions, including ankylosing spondylitis, multiple sclerosis, Crohn's disease, psoriasis, psoriatic arthritis, rheumatoid arthritis, and scleroderma. The most common chemotherapy agents act by killing cells that divide rapidly, one of the main properties of most cancer cells. This means that chemotherapy also harms cells that divide rapidly under normal circumstances: cells in the bone marrow, digestive tract, and hair follicles.

Chemotherapeutic compounds according to the present invention may be any one of alkylating agents, anti-metabolites, plant alkaloids, terpenoids, topoisomerase inhibitors (type I and II), and cytotoxic antibiotics.

Systemically administered chemotherapeutics are known to enter the pulmonary system and potentially cause local damages to the lungs, especially so in view of the compromised pulmonary host defense system as described herein elsewhere.

### Consequences of Pulmonary Dysfunction

The present invention provides use of granulocyte-macrophage colony-stimulating factor (GM-CSF), or a functional variant or homologue thereof, for treatment, prevention or alleviation of radiation-induced or chemotherapeutic-induced pulmonary dysfunction.

In one embodiment, the radiation-induced or chemotherapeutic-induced pulmonary dysfunction causes acute pulmonary dysfunction.

In one embodiment, the radiation-induced or chemotherapeutic-induced pulmonary dysfunction causes or is caused by pulmonary tissue injuries.

In response to irradiation or chemotherapy a reduced pulmonary immunological host defense against pulmonary infections occur.

Thus, it is an object to provide GM-CSF or a functional variant or homologue thereof for increasing the pulmonary host defense and consequently prevent, treat and/or reduce the risk of pulmonary infections associated with treatment with irradiation and/or chemotherapy.

In one embodiment, said reduced pulmonary immunological host defense causes or increases the risk of acquiring pulmonary infections with bacterial, fungal and/or viral infection or colonization of the lungs.

The present invention thus provides GM-CSF or a functional variant or homologue thereof for use in the treatment, prevention or alleviation of pulmonary infections associated with irradiation and/or chemotherapy. Said pulmonary infections may be selected from the group consisting of pneumonia of any kind, pneumonia with bacterial, fungal and/or viral infection or colonization including but not limited to pneumocystis carinii pneumonia, community acquired pneumonia, nosocomial pneumonia or ventilator associated pneumonia; cystic fibrosis with bacterial, fungal and/or viral infection or colonization; bronchitis with bacterial, fungal and/or viral infection or colonization; Bronchiectasis with bacterial, fungal and/or viral infection or colonization; Bronchiolitis with bacterial, fungal and/or viral infection or colonization including Diffuse panbronchiolitis, Bronchiolitis obliterans, Bronchiolitis obliterans organizing pneumonia (BOOP) with bacterial, fungal and/or viral infection or colonization

### EXAMPLES

### Example 1

### Prophylactic Therapy

A patient 64 yrs old presenting with non Hodgkin lymphoma earlier treated with high dose chemotherapy and subsequent allogenous bonemarrow transplantation. The patient is now referred to radiation therapy towards the mediastinum. At time of radiation therapy there were productive coughing and purulent sputum. It is decided to administer GM-CSF via inhalation of a daily dose of 300 microgram (morning and evening) for four days as preemptive intervention via a micropump nebulizer.

The patient was also administered an antibiotic systemically.

After completion of the radiation therapy, the combined GM-CSF inhalation and systemically administered antibiotic therapy was successful in as much as there were no signs and symptoms of pneumonia.

### Example 2

### Therapeutic Therapy

A middle-aged patient diagnosed with lung cancer presenting with pneumonia with bacterial infection after radiation therapy to the lungs.

Inhalation of GM-CSF via a micropump nebulizer at a dose of 300 microgram ×1 daily for 14 days.

The pulmonary host defense is increased by increasing the number of alveolar macrophages and by enhancing the autocrine function on the alveolar macrophages in order to transform the resting alveolar macrophages to fully immune-competent cells The early or manifest signs and symptoms of pneumonia are effectively treated.

### Example 3

Preemptive treatment should be initiated after suspected exposure of a radiation dose of at least <2 Gy by prompt dosing of 250-400 μg GM-CSF/m² or 5 μg/kg G-CSF administered systemically and concomitant inhalation of GM-CSF <300 mcg per day for at least 14-21 days.

The present United States standard for prevention and treatment of ARS standard intervention should consequently be modified into the combined systemic administration of growth factors and inhaled GM-CSF to ensure the sustained systemic and pulmonary host defense and thus prevent pulmonary dysfunction.

### Items

1. A method for inhibiting or alleviating radiation- or chemotherapeutic-induced effects in a subject in need thereof, said method comprising administering to the subject a composition comprising a selected cytokine.
2. The method of item 1, wherein the cytokine comprises granulocyte-macrophage colony-stimulating factor (GM-CSF), macrophage colony-stimulating factor (M-CSF), granulocyte colony-stimulating factor (G-CSF), stem cell factor (SCF), and/or an interleukin series (IL-1-IL-16).
3. The method of item 2, wherein the composition comprises GM-CSF.
4. The method of item 2, wherein the composition comprises M-CSF.
5. The method of item 2, wherein the composition comprises G-CSF.
6. The method of any of items 1 through 5, wherein the composition is administered locally.
7. The method of any of items 1 through 6, wherein the GM-CSF and/or M-CSF is pegylated.
8. The method of any of items 1 through 7, wherein the composition is administered as a liposomal formulation.
9. The method of any of items 1 through 8, wherein the composition is administered to a subject suffering from acute radiation syndrome.
10. The method of items 1 through 9 wherein the composition is administered by inhalation.
11. The method of any of items 1 through 10, wherein the composition is administered to a subject prior to and/or during radiation therapy.
12. The method of any of items 1 through 11, wherein the composition is administered to a subject prior to and/or during chemotherapy.

1-23. (canceled)

24. A method of alleviating radiation or chemotherapy-induced pulmonary dysfunction in a subject comprising:
administering to the subject locally by inhalation a composition comprising a mature fragment of granulocyte-macrophage colony-stimulating factor (GMCSF).

25. The method according to claim 24, wherein a dose of the inhaled GMCSF is 50 μg to 500 μg per m² of body surface area given once or twice daily.

26. The method according to claim 24, wherein the composition is administered to the subject prior to or and/or during and/or after radiation therapy.

27. The method according to claim 24, wherein the composition is administered prior to and/or during and/or after chemotherapy.

28. The method according to claim 24, wherein the GMCSF is administered by intratracheal, intrabronchial, intraalveolar or bronchoalveolar routes.

29. The method according to claim 24, wherein the GMCSF is to be administered as a solution, a suspension, an aerosol, a nebulized solution or a nebulized suspension.

30. The method according to claim 24, wherein said pulmonary dysfunction is an acute pulmonary dysfunction.

31. The method according to claim 24, wherein said radiation-induced pulmonary dysfunction causes a reduced pulmonary immunological host defense against pulmonary infection.

32. The method according to claim 31, wherein said pulmonary infection is associated with bacterial, fungal and/or viral infection and/or colonization of the lungs.

33. The method according to claim 24, wherein said radiation-induced pulmonary dysfunction is due to acute radiation syndrome (ARS).

34. The method according to claim 24, wherein said radiation-induced pulmonary dysfunction is due to radiation therapy.

35. The method according to claim 34, wherein said radiation therapy is targeted at the thorax and/or the lungs, and/or wherein said radiation therapy targets cancerous tissues of the thorax and/or the lungs.

36. The method of claim 24, wherein said mature fragment of GMCSF consists of amino acids 18 to 144 of SEQ ID NO: 1.

37. The method of claim 24, wherein said mature fragment of GMCSF consists of a protein having 95% sequence identity with amino acids 18 to 144 of SEQ ID NO: 1.


SEQUENCE LISTING (1 sequence)

SEQ ID NO: 1 - protein, 144 aa, Homo Sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE